(19) United States
(12) Patent Application Publication
(10) Pub. No.: US 2024/0343686 A1
(43) Pub. Date: Oct. 17, 2024

(54) FLOW SYNTHESIS PROCESS FOR THE PRODUCTION OF SULFONYLUREA COMPOUNDS

(71) Applicant: NELSON MANDELA UNIVERSITY, Port Elizabeth (ZA)

(72) Inventors: Agnes Zvinaiye KHASIPO, Port Elizabeth (ZA); Cloudius Ray SAGANDIRA, Port Elizabeth (ZA); Paul WATTS, Port Elizabeth (ZA)

(21) Appl. No.: 18/683,277

(22) PCT Filed: Aug. 12, 2022
(86) PCT No.: PCT/IB2022/057541

§ 371 Date: Feb. 13, 2024

(30) Foreign Application Priority Data
Aug. 13, 2021 (GB) 2111678.5

(51) Int. Cl. C07C 315/04; C07D 241/24

(52) U.S. Cl. CPC C07C 315/04; C07D 241/24; C07C 2601/14; C07C 2601/16

(57) ABSTRACT

This invention provides for a flow synthesis process for producing sulfonylurea compounds of formula (1), including glipizide and glibenclamide, and pharmaceutically acceptable salts thereof.

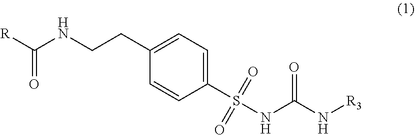

### INTRODUCTION

This invention relates to a process for producing sulfonylurea compounds, including glipizide and glibenclamide, and pharmaceutically acceptable salts thereof, in particular to a flow synthesis process for producing these compounds.

### BACKGROUND

Sulfonylureas, discovered by Janbon and co-workers in 1942, have been extensively used for treatment of type 2 diabetes for nearly 50 years. Despite several anti-diabetic agents on the market, sulfonylureas remain one of the most prescribed due to affordability, possibility of mono-dosing and presence of an association with metformin in the same tablet.

Diabetes mellitus is an endocrinological disorder that causes high blood sugar. According to the International Diabetes Federation (IDF) 425 million people had diabetes in 2017 worldwide with 1.8 million cases being reported in South Africa. Furthermore, it is predicted that about 552 million people worldwide will have diabetes by 2030.

Glipizide 1a and glibenclamide 1b are two second generation sulfonylurea drugs used for the treatment of diabetes mellitus. Although there are various synthetic approaches in literature towards these drugs, these processes are all inefficient.

Further, existing synthesis methodologies for the production of these compounds have essentially been based on standard stirred batch reactor type processes, wherein significant volumes of organic solvents are used.

Micro reactor technology (MRT), more recently branded ‘flow chemistry’, is an emerging technique that enables those working in research and development to rapidly screen reactions utilising continuous flow, leading to the identification of reaction conditions that are suitable for use at a production level. Furthermore, in addition to using conventional reaction methodology, the inherent safety associated with the use of small reactor volumes enables users to employ reaction conditions previously thought to be too hazardous for use within a production environment; such as extreme reaction conditions or the use/generation of ‘hazardous’ compounds. Consequently, the type of reactions available to the chemist increases through the use of this technology.

Although it is known to be desirable to transfer batch processes to flow synthesis, it often requires significant reaction condition and reagent modifications which are not necessarily obvious, considering the vast amount of permutations in reagents and reaction conditions and the unpredictable nature of chemistry in general. Furthermore, flow synthesis methodology has inherent challenges that has to be overcome, in particular for continuous multistep reactions.

The present invention seeks to address some of the shortcomings of the prior art by providing new flow chemistry processes for producing sulfonylurea compounds, including glipizide and glibenclamide.

### SUMMARY OF THE INVENTION

According to a first aspect to the present invention there is provided a flow synthesis process for producing a sulfonylurea compound of the Formula 1 or its pharmaceutically acceptable salts,

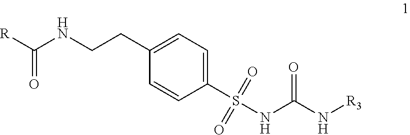

the process comprising the steps of:
   a) preparing the amide of Formula 2

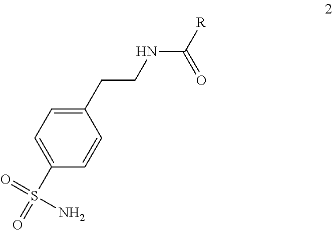

i) by activating a carboxylic acid of Formula 9 with a haloformate of Formula 8 in the presence of an organic base to produce an anhydride of Formula 5

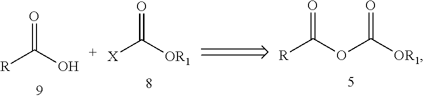

and
ii) reacting the anhydride of Formula 5 with the sulfonamide of Formula 4 to produce the amide of Formula 2,

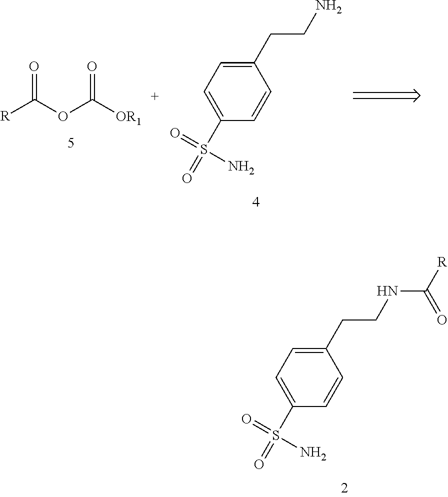

b) reacting the amide of Formula 2 with a carbamate of Formula 3 or isocyanate R₃—NCO,

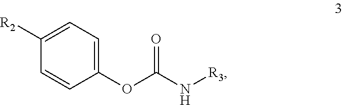

wherein the carbamate of Formula 3 is prepared through the reaction of R₃—NH₂ with the haloformate of Formula 7

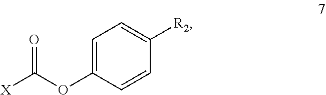

wherein:
R is selected from aryl or heteroaryl, which is unsubstituted or substituted with one or more occurrences of halogen, alkyl, and alkoxy;
R₁ is selected from phenyl, ethyl, or p-PhNO₂;
R₂ is selected from hydrogen and NO₂;
R₃ is a cyclohexyl; and
X is selected from Cl, Br, and I.

In one embodiment, the process is a continuous multi-step process without the isolation of any intermediates.

In one embodiment, R is selected from aryl or heteroaryl, which is substituted with one or more occurrences of halogen, alkyl, and alkoxy.

In one embodiment, R is selected from aryl or heteroaryl, which is substituted with one or more occurrences of chloro, methyl, and methoxy.

In one embodiment, R is selected from

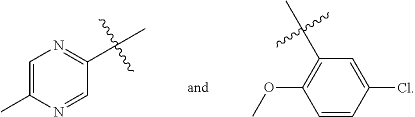

Preferably, X is Cl.

Preferably, the organic base in step (a)(i) is selected from TBA, DBU, DIPEA, THA, TEA and mixtures thereof.

In a preferred embodiment, the carboxylic acid of Formula 9 is dissolved in DMF.

In a preferred embodiment, the haloformate of Formula 8 is dissolved in acetonitrile.

Preferably, the reaction of step (a)(i) is thermally controlled to a temperature equal to about room temperature or less.

More preferably, the reaction of step (a)(i) is thermally controlled to a temperature of about room temperature.

In a preferred embodiment, the sulfonamide of Formula 4 is dissolved in DMF.

Preferably, the reaction of step (a)(ii) is thermally controlled to a temperature equal to about room temperature or less.

More preferably, the reaction of step (a)(ii) is thermally controlled to a temperature of about room temperature.

Preferably, the reaction of the amine R₃—NH₂ with a haloformate of Formula 7 in step (b) proceeds in the presence of an organic base selected from TBA, DBU, DIPEA, THA, TEA, and mixtures thereof.

More preferably, the organic base is either TBA or DBU.

In a preferred embodiment, the reaction of the amine R₃—NH₂ with a haloformate of Formula 7 in step (b) is thermally controlled to a temperature equal to about 100° C. or less.

More preferably, the reaction is thermally controlled to a temperature of about room temperature.

Preferably, the reaction of the amide of Formula 2 with a carbamate of Formula 3 or isocyanate R₃—NCO in step (b) proceeds in the presence of an organic base selected from TBA, DBU, DIPEA, THA, TEA, and mixtures thereof.

Preferably, the organic base is DBU.

Preferably, the amide of Formula 2 and the organic base is provided at a molar equivalent ratio of amide:base of about 1:1 to about 1:5.

More preferably, the amide of Formula 2 and the organic base is provided at a molar equivalent ratio of amide:base of about 1:2 to about 1:3.

In one embodiment, in the reaction of step (b) the amide of Formula 2 is dissolved in a solvent selected from DMF, acetonitrile, and mixtures thereof, and the carbamate of Formula 3 is dissolved in chloroform.

In one embodiment, in the reaction of step (b) the amide of Formula 2 is provided at a concentration between about 0.01 M and about 0.5 M.

Preferably, the reaction of step (b) is thermally controlled to a temperature between about room temperature and about 150° C.

More preferably, the reaction of step (b) is thermally controlled to a temperature of about 80° C.

In a preferred embodiment, the compound of the Formula 1 is selected from the compounds of Formula 1a or 1b and pharmaceutically acceptable salts thereof,

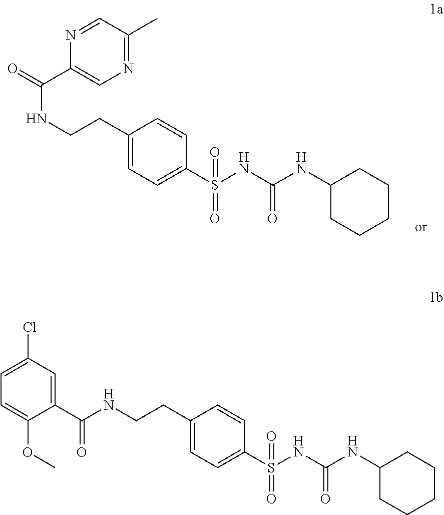 [SMILES: Cc1cnc(C(=O)NCCc2ccc(S(=O)(=O)NC(=O)NC3CCCCC3)cc2)cn1 ; COc1ccc(Cl)cc1C(=O)NCCc1ccc(S(=O)(=O)NC(=O)NC2CCCCC2)cc1]

### BRIEF DESCRIPTION OF THE DRAWINGS

The invention will now be described in more detail with reference to the following non-limiting embodiments and figures in which:

FIG. 1 shows the effect of residence time on carboxylic acid 9a activation;

FIG. 2 shows a comparison of the conversion to sulfonylurea 1 using various DMF:ACN solvent mixtures; and

FIG. 3 shows the effect of residence time on the continuous flow synthesis of sulfonylurea 1 using isocyanate 10.

### DETAILED DESCRIPTION OF A PREFERRED EMBODIMENT

The present invention will now be described more fully hereinafter with reference to the accompanying figures, in which some of the non-limiting embodiments of the invention are shown.

The invention as described hereinafter should not be construed to be limited to the specific embodiments disclosed, with slight modifications and other embodiments intended to be included within the scope of the invention.

Although specific terms are employed herein, they are used in a generic and descriptive sense only and not for purposes of limitation.

As used herein, throughout this specification and in the claims which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having”, “including”, and variations thereof used herein, are meant to encompass the items listed thereafter, and equivalents thereof as well as additional items.

The present invention provides for a flow synthesis process for producing sulfonylurea compounds and pharmaceutically acceptable salts thereof, in particular to a flow synthesis process for producing glipizide and glibenclamide. In a particularly preferred embodiment, the invention provides for a continuous multistep flow synthesis process for producing glipizide and glibenclamide without the isolation of any intermediates.

Although it is known to be desirable to transfer exiting batch processes or methodologies to flow synthesis systems, it requires significant reaction condition and reagent type modifications which are not obvious to the skilled person, in particular considering the endless permutations in reagents and reaction conditions available. Furthermore, flow synthesis has inherent challenges to overcome, in particular for continuous multistep reactions. The inventors of the present invention not only overcame these challenges, but developed highly efficient and rapid methods for the synthesis of various sulfonylurea compounds, as disclosed herein.

As shown in Scheme 1 below, in one embodiment the invention provides for the preparation of sulfonylurea 1 (Glipizide 1a and Glibenclamide 1b) from amide of the formula 2 and carbamate of formula 3, or isocyanate of formula 10, as key intermediates. Amide 2 can be prepared from amidation of sulfonamide of formula 4 with the activated form of carboxylic acid of formula 9 (i.e. anhydride of formula 5). In parallel, carbamate of formula 3 can be prepared from the treatment of amine of formula 6 with chloroformate of formula 7.

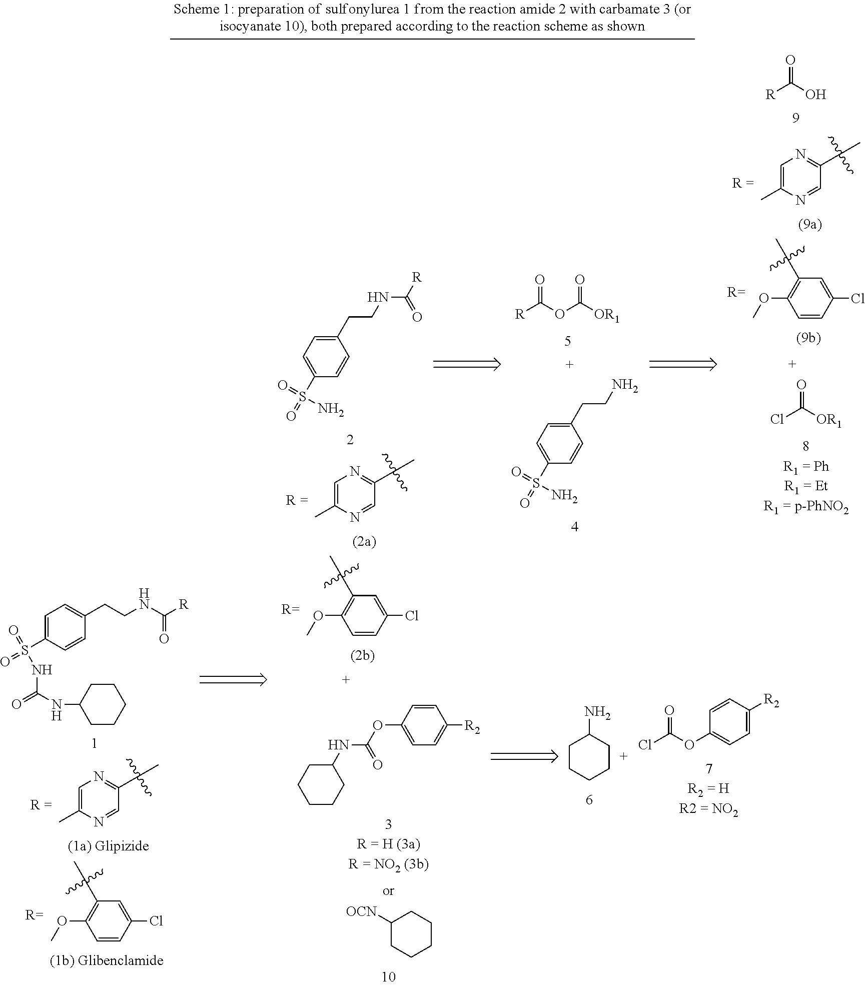

### General Experimental Procedures

Chemicals were supplied by Sigma Aldrich, Merck and Industrial Analytical and were used as received. Anhydrous solvents were supplied by Sigma Aldrich, and maintained by drying over appropriately activated molecular sieves during use.

Column chromatography was performed using Fluka Chemie silica gel 60 as the stationary phase, and mixtures of ethyl acetate and hexane of varying polarity were used as the mobile phase. Unless otherwise stated, thin layer chromatography (TLC) was done using Merck Kieselgel 60 HF254 aluminium backed TLC plates with mixtures of ethyl acetate and hexane of varying polarity as eluent. TLC visualisation was done by fluorescence on exposure to short wave ultra violet (UV) light (λ 254 nm) in a Camag UV cabinet.

Nuclear magnetic resonance (NMR) spectra were recorded at room temperature as solutions in deuterated chloroform (CDCl₃) or deuterated dimethyl sulfoxide (DMSO-d₆). A Bruker Avance-400 spectrometer (400 MHz) was used to record the spectra and the chemical shifts are reported in parts per million (ppm) with coupling constants in Hertz (Hz).

Infra-red spectra were recorded from 4000 to 500 cm⁻¹ using a Bruker spectrometer and peaks (Vₘₐₓ) reported in wavenumbers (cm⁻¹). Melting points of all compounds were determined using a Staurt Melting Point Apparatus SMP30.

High performance liquid chromatography (HPLC) data was obtained using Agilent 1220 with a UVNis detector: HPLC analysis was performed on ACE Generix 5 C18(2) column (150 mm×4.6 mm i.d) at ambient temperature using an isocratic system. The mobile phase consisted of 70% acetonitrile and 30% water. The sample injection volume was 1 μl, eluted at a flow rate of 1.5 ml/min and detected at 230 nm with a run time of 7 min.

The collected carbamate solutions were washed with water and extracted into DCM. The products were concentrated at room temperature under reduced pressure to afford a solid. For spectroscopic analysis, the products were washed with acetone and air dried. The products were characterized by FTIR and NMR.

The collected sulfonylurea solutions were concentrated under reduced pressure and redissolved in DCM and washed with water. The organic layer was concentrated under vacuum to afford a white solid. The products were purified with methanol and oven dried at 60° C. and characterized by FTIR and NMR.

### Reaction 1: Flow Synthesis Activation of Carboxylic Acid 9 Toward Anhydride 5

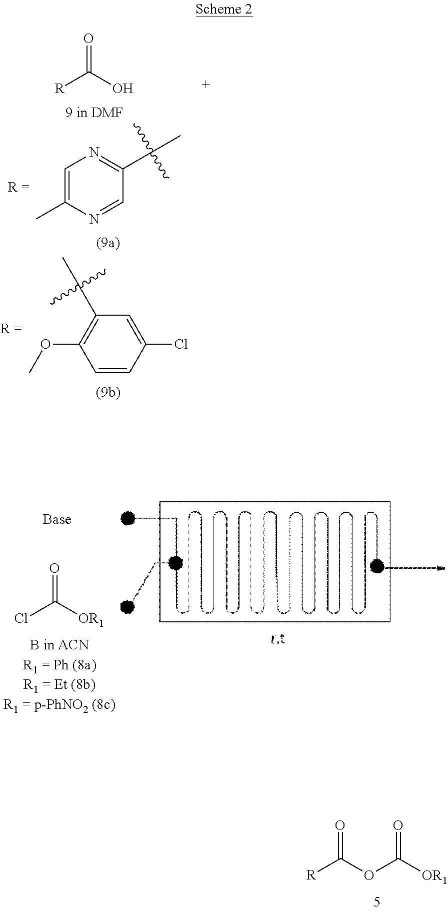

In one embodiment of the invention, as shown in Scheme 2 above, carboxylic acid 9 (9a and 9b) was activated in a flow process by the treatment with chloroformate 8 in the presence of a suitable organic base to afford anhydride 5. In one embodiment, a premix of carboxylic acid 9a (0.1 M, 1 Equiv.) and tributyl amine (“TBA”) (1 Equiv.) in dimethylformamide (“DMF”) was treated with phenyl chloroformate 8a (1 Equiv.) in acetonitrile (“ACN”) to afford the respective intermediate 5 at room temperature. It is envisaged that R may be selected from any aryl or heteroaryl group, and that these groups may be unsubstituted or substituted with one or more halogen, alkyl, or alkoxy groups. In one embodiment, R is an aryl or heteroaryl substituted with one or more chloro, methyl, and methoxy groups.

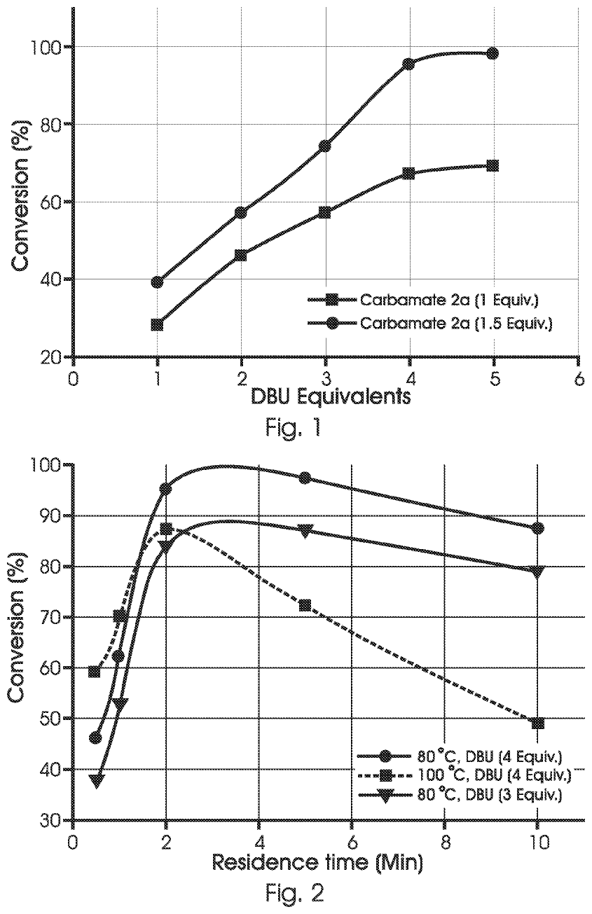

As can be seen from FIG. 1, carboxylic acid 9a activation generally improved with increase in residence time. Full conversion to the anhydride was achieved at a residence time of about 1.5 minutes. Further experiments were conducted to investigate carboxylic acid 9b activation, as well as the use of alternative bases and chloroformates (Ba, 8b and 8c). Alternative bases investigated included 1,8-Diazabicyclo(5.4.0)undec-7-ene (“DBU”), N,N-Diisopropylethylamine (“DIPEA”), (Trihexylamine) “THA”, and triethylamine (“TEA”).

TABLE 1
The investigation of carboxylic acid 9 activation in flow.

| | Carboxylic | | Chloro- | Conversion. |
|---|---|---|---|---|
| Entry | acid 9 | Base | formate 8 | (%)^{e} |
| 1 | 9a | TBA | 8a | 100 |
| 2 | 9a | TBA | 8b | 100 |
| 3 | 9a | TBA | 8c | 100 |
| 4 | 9a | Base^{b} | 8^{c} | 100 |
| 5 | 9b | TBA | 8a | 100 |
| 6 | 9b | Base^{b} | 8^{c} | 100 |
| 7^{a} | 9a | TBA | 8a | 100 |
| 8^{d} | 9b | TBA | 8a | 100 |

^{a}Standard conditions: Premix of carboxylic acid 9 (0.1M, 1 Equiv.) and base (0.1M, 1 equiv.) in DMF and chloroformate 8 (1 Equiv.) in ACN, 1.5 min residence time and r.t.
^{b}Base is DBU or DIPEA or THA or TEA
^{c}Chloroformate 8 is 8a or 8b or 8c
^{d}Premix of carboxylic acid 9 (3M, 1 Equiv.) and base (3M, 1 equiv.) in DMF and chloroformate 8 (1 Equiv.) in ACN, 1.5 min residence time and r.t.
^{e}Conversion determined by HPLC.

Activation of carboxylic acid 9a was successfully achieved by treatment with various chloroformate 8a to 8c in the presence of TBA to afford anhydride 5 in full conversion (Table 1, Entries 1-3). The use of alternative bases and chloroformates afforded respective anhydride 5 in full conversion (Table 1, Entry 4). Carboxylic acid 9b was activated in the presence of bases and chloroformates to afford respective anhydride 5 in full conversion (Table 1, Entries 5-6). Surprisingly, higher concentrations were successfully used without any complications (Table 1, Entries 7-8). Generally, carboxylic acid 9 activation was efficiently demonstrated in flow to afford anhydride 5 in full conversion.

### Reaction 2: Flow Synthesis Preparation of Amide 2 Via Anhydride 5 Formed In Situ

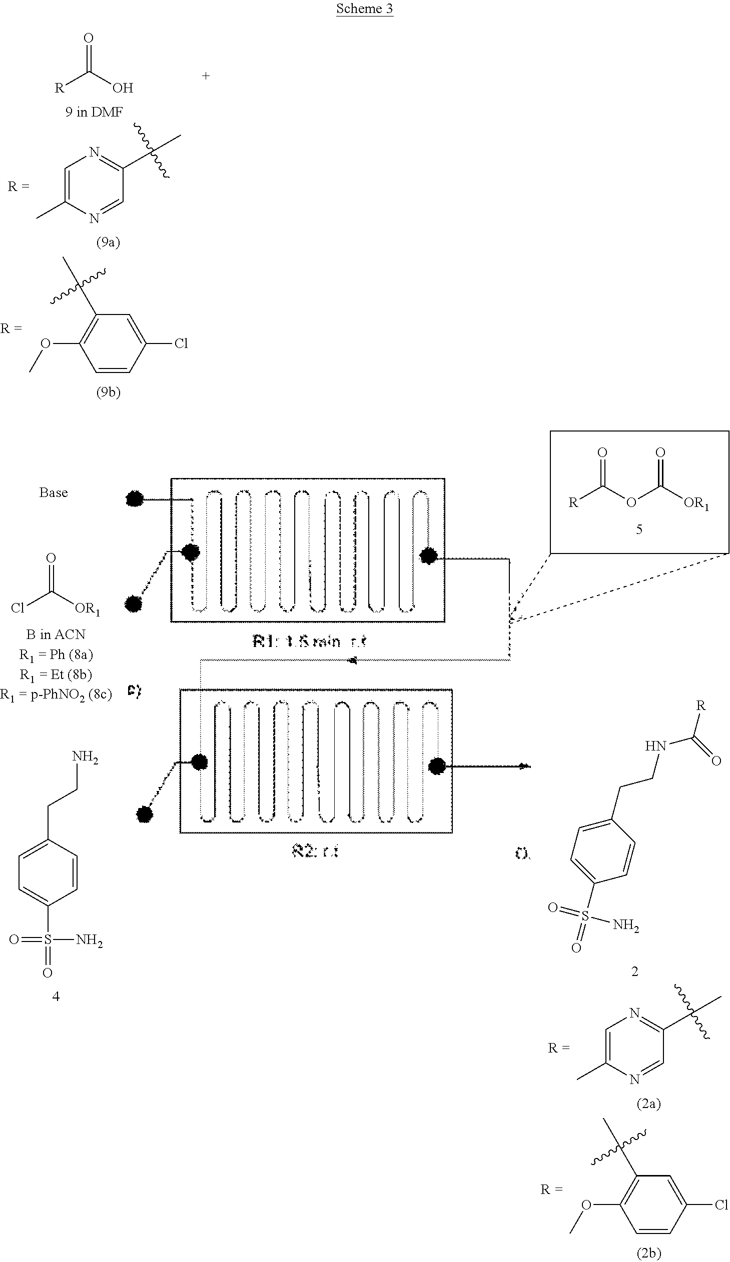

With the optimum conditions for anhydride 5 synthesis from carboxylic acid 9 determined, a two-step synthesis of amide 2 was investigated. This was achieved by in situ generation and consumption of anhydride 5 with sulfonamide 4 to afford amide 2. In one embodiment of the invention, a premix of carboxylic acid 9a (0.1 M, 1 Equiv.) and TBA (0.1 M, 1 Equiv.) in DMF was activated with chloroformate 8a (0.1 M, 1 Equiv.) in ACN at the predetermined optimum conditions (1.5 min residence time and room temperature) in the first reactor (R1, 2 ml) to afford anhydride 5 which was subsequently treated with sulfonamide 4 (0.5 M, 1 Equiv.) at room temperature for 0.75 min in the second reactor (R2, 2 ml) to afford amide 2a in full conversion (Scheme 3). Further investigations focused on the synthesis of amide 2b, and the use of various bases and chloroformates in the multistep synthesis of amide 2 (Scheme, Table 2).

TABLE 2
Two-step flow synthesis of amide 2 from carboxylic acid 9

| | | | | | R2 Res. | Conver- |
|---|---|---|---|---|---|---|
| | Carboxylic | | Chloroformate | Amide | Time | sion. |
| Entry | acid 9 | Base | 8 | 2 | (min) | (%)^{e} |
| 1 | 9a | TBA | 8a | 2a | 0.75 | 100 |
| 2 | 9b | TBA | 8a | 2b | 0.75 | 100 |
| 3 | 9a | Base^{b} | 8^{c} | 2a | 0.75 | 100 |
| 4 | 9b | Base^{b} | 8^{c} | 2b | 0.75 | 100 |
| 5^{d} | 9a | TBA | 8a | 2a | 0.75 | 100 |
| 6^{d} | 9b | TBA | 8a | 2b | 0.75 | 100 |
| 7^{d} | 9a | TBA | 8a | 2b | 0.5 | 100(98)^{f} |
| 8^{d} | 9b | TBA | 8a | 2b | 0.5 | 100(98)^{f} |
| 9^{d} | 9a | TBA | 8a | 2a | 0.25 | 74 |
| 10^{d} | 9b | TBA | 8b | 2b | 0.25 | 78 |

^{a}Standard conditions: Premix of carboxylic acid 9 (0.1M, 1 Equiv.) and base (0.1M, 1 equiv.) in DMF and chloroformate 8 (1 Equiv.) in ACN, Sulfonamide 4 (0.05M, 1 Equiv.) in DMF
^{b}Base is DBU or DIPEA or THA or TEA
^{c}Chloroformate 8 is 8a or 8b or 8c
^{d}Premix of carboxylic acid 9 (3M, 1 Equiv.) and base (3M, 1 equiv.) in DMF and chloroformate 8 (1 Equiv.) in ACN, Sulfonamide 4 (1.5M, 1 Equiv.) in DMF
^{e}Conversion determined by HPLC
^{f}Number in parenthesis is isolated yield.

The procedure developed for amide 2a synthesis (2.25 min total residence time; R1=1.5 min, R2=0.75 min), was used to synthesise amide 2b in full conversion (Table 2, Entries 1-2). Various bases (including TBA, DBU, DIPEA, THA, and TEA) and chloroformates were successfully used in the carboxylic acid 9 activation step of the multistep synthesis of amide 2 (Table 2, Entries 3-4). Surprisingly, the reaction concentration was increased successfully affording amide 2 in full conversion and excellent yield (Table 2, Entries 5-8). Amide 2 could also be synthesised in full conversion and excellent isolated yield in shorter residence time (2 min total residence time; R1=1.5 min, R2=0.5 min) (Table 2, Entries 7-8). However, further shortening of residence time was accompanied by a decrease in conversion (1.75 min total residence time; R1=1.5 min, R2=0.25 min) (Table 2, Entries 9-10). In a preferred embodiment of the invention, the optimum conditions for the synthesis of the two important intermediates for glipizide 1a and glibenclamide 1b (amide 2a and amide 2b respectively) in full conversion were found to be room temperature and 2 min total residence time (R1=1.5 min. R2=0.5 min).

In one embodiment of the invention, the procedure was scaled-up in a Kiloflow reactor (R1=6.1 ml (1.5 min), R2=12.2 ml (1.5 min)) where a premix of carboxylic acid 9 (3 M, 1 Equiv.) and TBA (3 M, 1 Equiv.) in DMF was activated with chloroformate 8a (3 M, 1 Equiv.) in ACN at room temperature in the first reactor for 1.5 min to afford anhydride 5 in situ which was subsequently treated with sulfonamide 4 (1.5 M, 1 Equiv.) at room temperature in the second reactor for 0.5 min to afford amide 2 in full conversion and 98% isolated yield with a throughput of 113-130 g/h. Amide 2 was vacuum filtered, washed with acetone, oven dried at 60° C. and product was characterized with NMR and FTIR.

Reaction 3: Continuous Flow Synthesis Preparation of Carbamate 3 from Amine 6 and Chloroformate 7

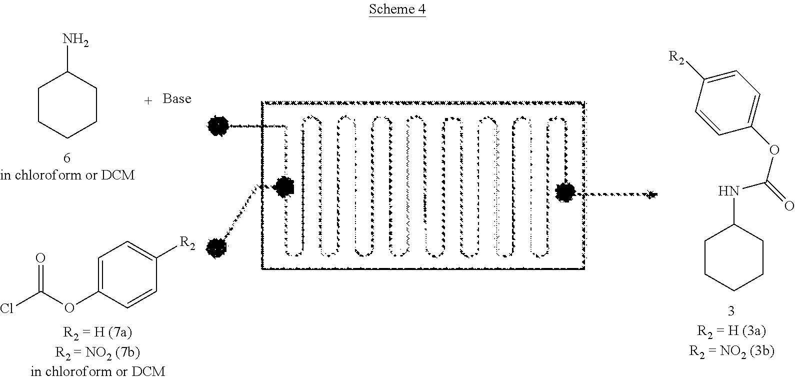

In one embodiment, carbamate 3 was prepared by treating amine 6 with chloroformate 7 in the presence of a suitable base in a 2 ml glass reactor to afford the desired carbamate 3 (FIG. 4, Table 3). Either chloroform or DCM was used as solvent with chloroform being the preferred solvent in this embodiment.

Carbamate 3a was prepared at 0° C. in full conversion (Table 3, Entry 1). An increase in temperature to room temperature afforded the same results (Table 3, Entry 2). However, a decrease in residence to 15 seconds at room temperature was accompanied with a decrease in conversion (Table 3, Entry 3).

Increasing temperature to 100° C. (at 15 s residence time) was accompanied by further decrease in conversion (Table 3, Entry 4). Chloroformate 7a and 7b together with DBU or TBA was used to afford the respective desired carbamate 7 in full conversion (Table 3, Entry 5). The use of a base was avoided using an excess of amine 6 to afford carbamate 3 in full conversion. An increase in concentration to 1 M successfully afforded carbamate 3 in full conversion and 97% isolated yield. Use of concentrations above 1 M was accompanied by blockage of the reactor in the specific reactor used in this embodiment. The optimum conditions, for this embodiment, were found to be amine 6 (1 M, 1 Equiv.), chloroformate 7 (1 Equiv.), base (DBU or TBA) (1 Equiv.), 30 s residence time and room temperature to afford carbamate 7 in full conversion and 97% isolated yield. The reaction can also proceed in the absence of base. In one embodiment of the invention, the procedure was scaled-up in a Kiloflow reactor (23.8 ml) to afford carbamate 7 in full conversion and 97% isolated yield with a throughput of 303-365 g/h. The collected carbamate 3 solution was washed with water and extracted into DCM. The product was concentrated at room temperature under reduced pressure to afford a solid. For spectroscopic analysis, the product was washed with acetone and air dried. The product was characterized with NMR and FTIR.

TABLE 3
Investigation of carbamate 3 synthesis in flow^{a}

| | Res. | | Chloro- | | |
|---|---|---|---|---|---|
| | Time | Temp. | formate | | Conversion |
| Entry | (s) | (° C.) | 7 | Base | (%)^{d} |
| 1 | 30 | 0 | 7a | DBU | 100 |
| 2 | 30 | r.t | 7a | DBU | 100 |
| 3 | 15 | r.t | 7a | DBU | 89 |
| 4 | 15 | 100 | 7a | DBU | 75 |
| 5 | 30 | r.t | 7a or 7b | DBU or TBA | 100 |
| 6^{b} | 30 | r.t | 7a or 7b | No base | 100 |
| 7^{c} | 30 | r.t | 7a or 7b | DBU or TBA | 100 (97%)^{e} |

^{a}Standard conditions: Amine 6 (0.1M, 1 Equiv.), Base (1 Equiv.) and chloroformate 7 (1 Equiv.),
^{b}Amine 6 (0.1M, 2 Equiv.), No base, chloroformate 7a (1 Equiv.),
^{c}Amine 6 (1M, 1 Equiv.), DBU (1 Equiv.), chloroformate 7a (1 Equiv.),
^{d}Conversion determined by HPLC,
^{e}Number in parentheses corresponds to isolated yield.

### Reaction 4: Flow Synthesis Preparation of Sulfonylurea 1 Via Carbamate 3

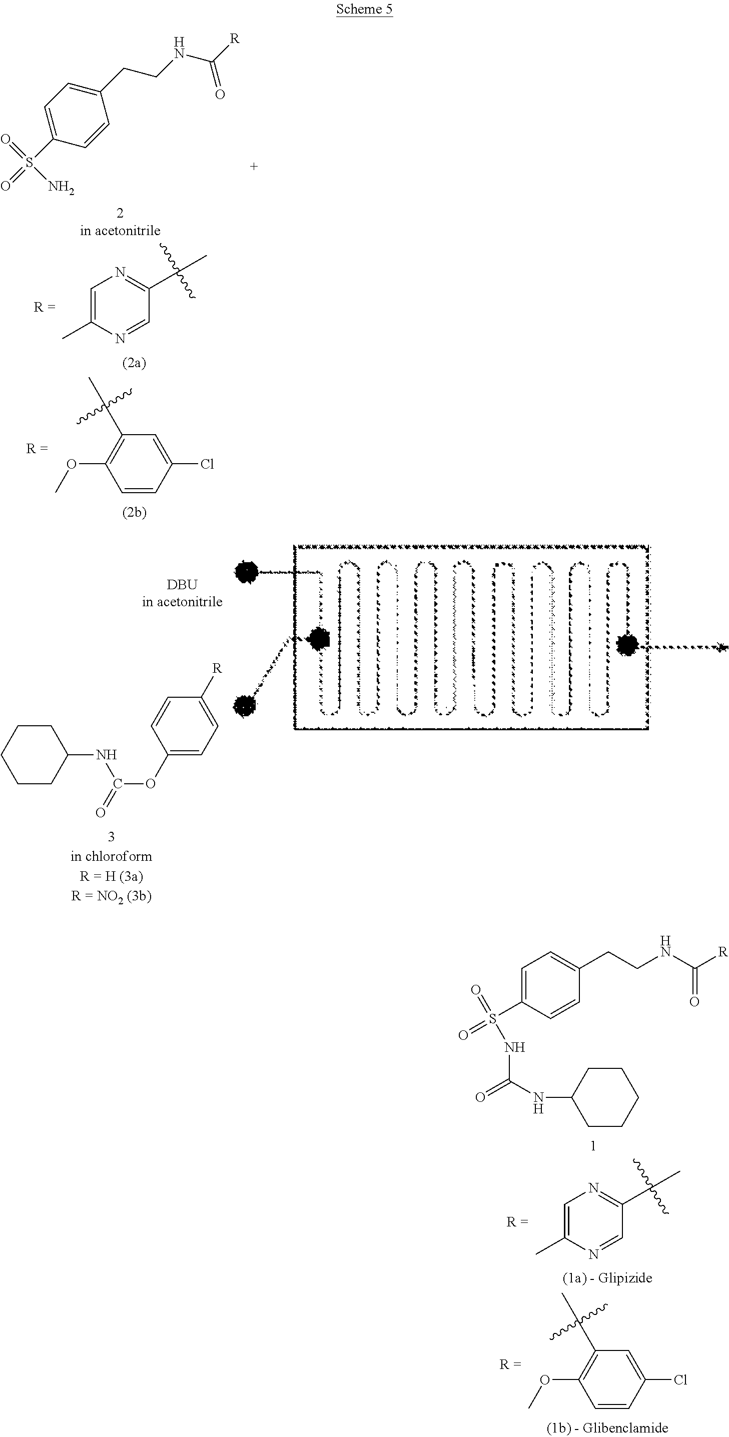

Sulfonylurea 1 (glipizide 1a and glibenclamide 1b) was prepared by treating amide 2 with carbamate 3. Due to poor solubility of amide 2 in most solvents, the experiments were done at relatively low concentrations. Amide 2 (0.01 M, 1 Equiv.) premixed with DBU (1 Equiv.) in ACN was treated with carbamate 3 (1 Equiv.) in chloroform to afford sulfonylurea 1. Initial experiments were performed in a 2 ml reactor (Scheme 5, Table 4).

TABLE 4
Investigation of the flow synthesis of sulfonylurea
1 from amide 2 and carbamate 3

| | | Res. | | | | Conver- |
|---|---|---|---|---|---|---|
| | Amide | Time | Temp. | Chloroformate | DBU | sion |
| Entry | 2 | (min) | (° C.) | 7 Equiv. | Equiv. | (%)^{d} |
| 1 | 2a | 2 | 80 | 1 | 2 | 63 |
| 2 | 2a | 5 | 80 | 1 | 2 | 68 |
| 3 | 2a | 10 | 80 | 1 | 2 | 68 |
| 4 | 2a | 5 | 150 | 1 | 2 | 62 |
| 5 | 2a | 5 | 80 | 1.2 | 2 | 85 |
| 6 | 2a | 5 | 80 | 1.2 | 3 | 97 |
| 7 | 2b | 5 | 80 | 1.2 | 3 | 98 |
| 8 | 2b | 10 | 80 | 1.2 | 3 | 98 |
| 9^{b} | 2a | 5 | 80 | 1.2 | 3 | 97 (93) |
| 10^{c} | 2b | 5 | 80 | 1.2 | 3 | 98 (95) |

^{a}Amide 2 (0.01M, 1 Equiv.) premixed with DBU in ACN, carbamate 7 in chloroform,
^{b}Amide 2a (0.1M, 1 Equiv.) premixed with DBU (3 Equiv.) in ACN, carbamate 3 in chloroform,
^{c}Amide 2b (0.5M, 1 Equiv.) premixed with DBU (3 Equiv.) in ACN, carbamate 7 in chloroform,
^{d}Conversion determined by HPLC.

Performing the reaction at 80° C. with 2 min residence time afforded glipizide 1 in 63% conversion (Table 4, Entry 1). An increase in residence time increased conversion to a point where it remained constant (Table 4, Entries 1-3). Performing the reaction at higher temperature (150° C.) was accompanied by a decrease in conversion (Table 4, Entry 4). An increase in the equivalents of both DBU and carbamate 3 resulted in a surprising improvement in conversion (Table 4, Entries 5-6). In one embodiment of the invention, the preferred conditions for glipizide 1a synthesis were found to be 80° C. and 5 min residence time to afford 97% conversion (Table 4, Entry 6). At these preferred conditions for glipizide 1a synthesis, glibenclamide 1b was prepared in 98% conversion (Table 4, Entry 7). Doubling the residence time did not improve conversion (Table 4, Entry 8). Due to poor solubility of amide 2 in ACN, the maximum concentrations used were 0.1 M and 0.5 M for amide 2a and 2b, respectively, in the presence of DBU to afford glipizide 1 and glibenclamide 1b in 97% (93 isolated yield) and 98% (95 isolated yield) conversion respectively (Table 4, Entries 9-10).

As amide 2 has better solubility in DMF, its use was investigated using the otherwise the same reaction conditions (FIG. 2). As can be seen from Figure, interestingly, the use of ACN afforded the best results and the use of DMF gave the worst results. Without thereby wishing to be bound by any particular theory, it is believed that the poor results associated with use of DMF could possibly be attributed to its nucleophilicity, as it may react with the isocyanate intermediate formed from carbamate 7. The use of solvent mixtures (DMF/ACN) were investigated to establish whether a compromise between DMF's good solvency towards amide 2 and its undesired nucleophilic properties would be possible. The conversion towards sulfonylurea 1 decreased with an increase in the proportion of DMF in the DMF/ACN solvent mixture. Although reaction concentrations can be increased by incorporating DMF due to its good solvency towards amide 2, it is accompanied by a decrease in conversion towards the desired sulfonylurea 1.

With the preferred conditions from the previous embodiment in hand, amide 2a (0.1 M, 1 Equiv.) premixed with DBU (3 Equiv.) in ACN, carbamate 3 in chloroform, 80° C. and 5 min residence time (for sulfonylurea 1a), and amide 2b (0.5 M, 1 Equiv.) premixed with DBU (3 Equiv.) in ACN, carbamate 3 in chloroform, 80° C. and 5 min residence time (for sulfonylurea 1b) were scaled-up in a Kiloflow reactor (18.4 ml) to afford sulfonylurea 1 (glipizide 1a and glibenclamide 1b) in 96-98% conversion and 93-95% isolated yield with a throughput of 4.6-26 g/h. After reaction completion, product was washed with aq. HCl (0.1 M), extracted into DCM concentrated under vacuum to get a solid product which was further purified with methanol. The product was characterized with NMR and FTIR.

### Reaction 5: Two Step Flow Synthesis Preparation of Sulfonylurea 1

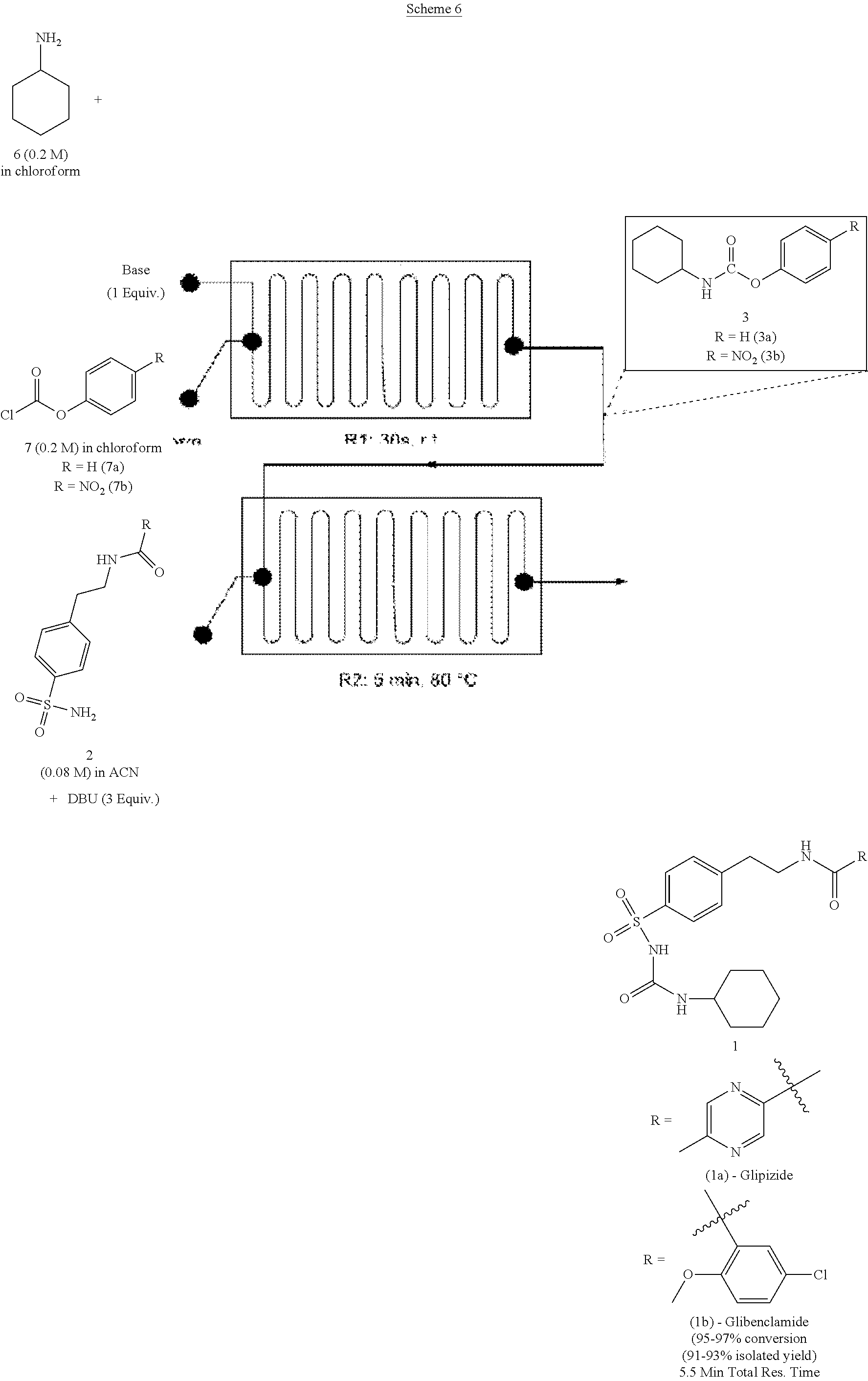

With the preparation of the two important intermediates for sulfonylurea 1 (amide 2 and carbamate 3) developed in flow, a multistep synthesis of sulfonylurea 1 from amine 6 was performed (Scheme 6).

Amine 6 (0.2 M, 1 Equiv.) was treated with chloroformate 7 at room temperature in the first reactor (R1) for 30 s to afford carbamate 3 in situ which was subsequently treated with amide 2 (0.1 M, 1 Equiv.) premixed with DBU (3 Equiv.) at 80° C. for 5 min in the second reactor (R2) to afford sulfonylurea 1 in 95-97% conversion (91-93% isolated yield). After reaction completion, the product solution was washed with aq. HCl (0.1 M), extracted into DCM concentrated under vacuum to get a solid product which was further purified with methanol and oven dried 60° C. The product was characterized with NMR and FTIR.

### Reaction 6: Flow Synthesis Preparation of Sulfonylurea 1 Via Isocyante 10

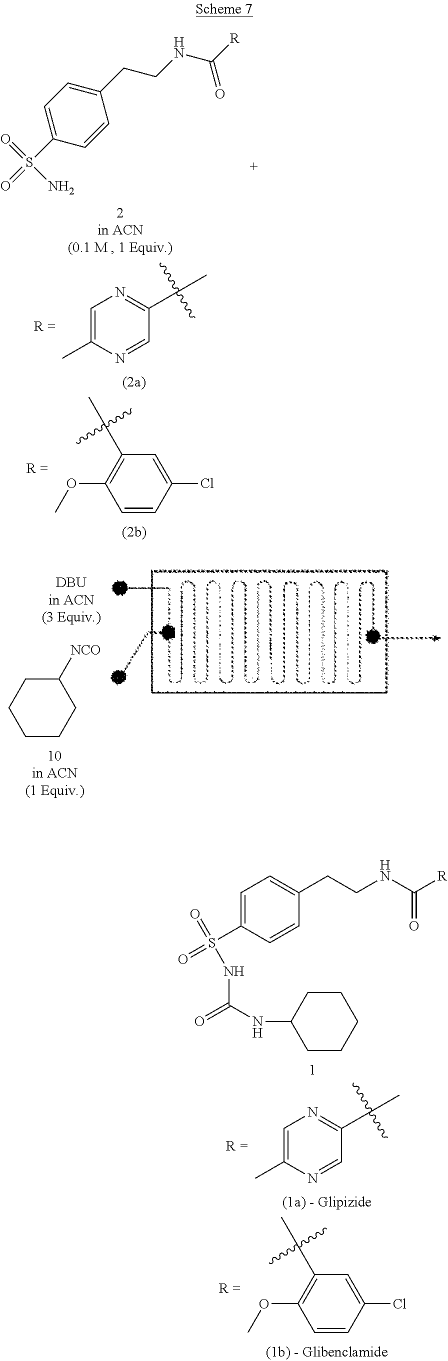

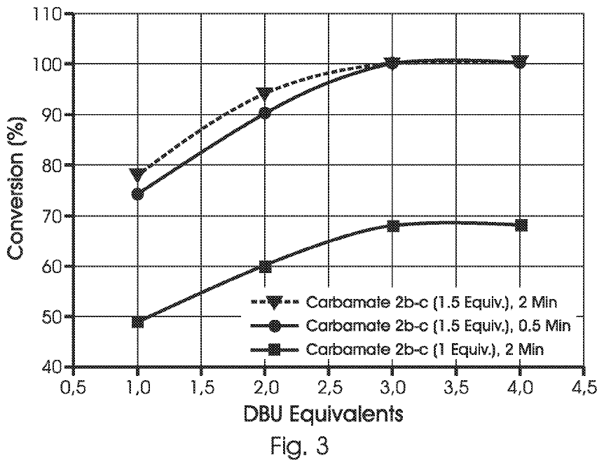

In an alternative embodiment of the invention, amide 2 was treated with commercially available isocyanate 10 to afford sulfonylurea 1. The reaction was investigated in a 2 ml flow reactor (Scheme 7). Synthesis of sulfonylurea 1 improved with increase in residence time and temperature (FIG. 3). In this embodiment, preferred conditions were found at 100° C., 2 min residence time to afford sulfonylurea 1 in full conversion.

### Reaction 7: Multistep Flow Synthesis Preparation of Sulfonylurea 1 Via Amide 2 and Carbamate 3 Formed In Situ

Multistep synthesis of sulfonylurea 1 was attempted even though the inventors foresaw that % conversion may be less desirable compared to a process which is not fully integrated. This is so as a result of the incompatibility of DMF used in step 1 with the reaction in step 2. In this embodiment, shown in Scheme 8 below, multistep synthesis produced sulfonylurea 1 in 31-33% conversion. However, it is anticipated that this multistep method could still be commercially desirable with the requisite optimisations.

Carboxylic acid 9 (2 M, 1 Equiv.) premixed with base (1 Equiv.) in DMF was treated with chloroformate 8 (1 Equiv.) in ACN in the first reactor at room temperature for 1.5 min to afford anhydride 5 which was subsequently treated with sulfonamide 4 (1 M, 1 Equiv.) in DMF in the second reactor at room temperature for 0.5 min to afford amide 2 which was subsequently treated with carbamate 3 (prepared in parallel in the third reactor held at room temperature for 0.5 min from the treatment of chloroformate 7 (0.5 M) with amine 6 (0.5 M, 1 Equiv.) premixed with base (1 Equiv.)) in the fourth reactor held at 80° C. for 5 min to afford sulfonylurea 1 (Scheme 8).

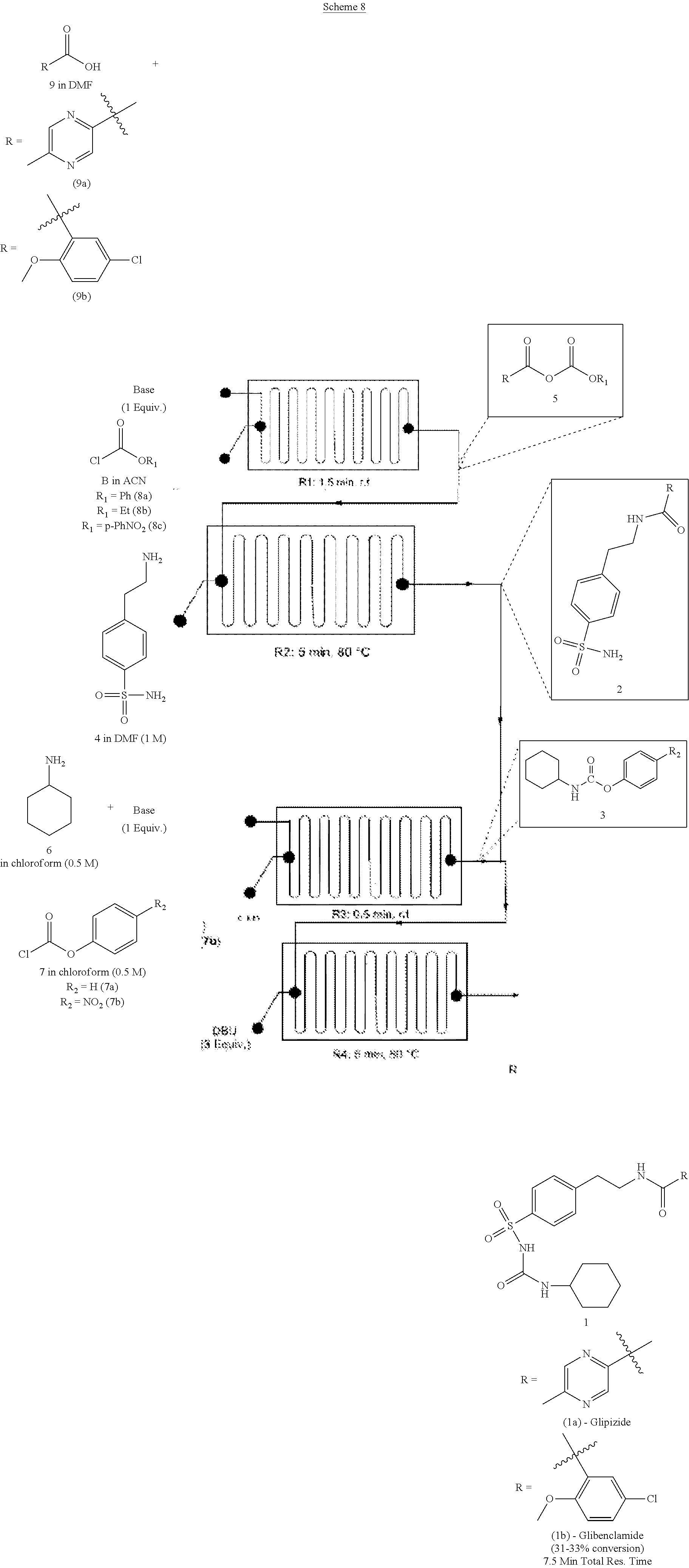

### Spectroscopic Data

### Amide 2a

Amide 2a was afforded as a yellowish solid, mp 210-213° C.; ¹H NMR (400 MHz, DMSO-d₆) δ 9.08 (s, 1H), 8.99 (t, J=6.1 Hz, 1H), 8.65 (s, 1H), 7.79 (d, J=7.9 Hz, 2H), 7.48 (d, J=7.9 Hz, 2H), 7.34 (s, 2H), 3.63 (q, J=6.9 Hz, 2H), 3.01 (t, J=7.3 Hz, 2H), 2.64 (s, 3H). ¹³C NMR (101 MHz, DMSO-d₆) δ 163.0, 156.8, 143.5, 142.8, 142.4, 142.5, 129.4, 125.7, 126.1, 35.4, 21.8. FTIR (cm⁻¹) v: 3267.9, 1651.6, 1537.9, 1475, 1327.2, 1273.8, 1156.1, 1095.7, 1037.3, 913, 840.4, 685.5, 589.2, 542.3.

### Amide 2b

Amide 2b was afforded as a white solid, mp 180-183° C.; ¹H NMR (400 MHz, DMSO-d₆) δ 8.27 (t, J=5.7 Hz, 1H), 7.78 (d, J=7.9 Hz, 2H), 7.66 (d, J=2.8 Hz, 1H), 7.51 (dd, J=8.9, 2.7 Hz, 1H), 7.46 (d, J=7.9 Hz, 2H), 7.31 (s, 2H), 7.16 (d, J=8.9 Hz, 1H), 3.82 (s, 3H), 3.54 (q, J=6.7 Hz, 2H), 2.92 (t, J=7.1 Hz, 2H). ¹³C NMR (101 MHz, DMSO-d₆) δ 163.6, 155.6, 143.6, 142.1, 131.6, 129.4, 129.6, 126.1, 125.3, 124.8, 114.6, 56.7, 40.3, 34.0. FTIR (cm⁻¹) v: 3313.4, 1716.9, 1631.3, 1538.1, 1481.7, 1321.6, 1272.5, 1183, 1159.6, 1094, 1022.6, 807.7, 644.9, 585.4, 551.4.

### Carbamate 3a

Carbamate 3a was afforded as a white solid, mp 138-140° C.; ¹H NMR (400 MHz, CDCl₃) δ 7.25 (t, J=7.7 Hz, 2H), 7.09 (d, J=7.4 Hz, 1H), 7.04 (d, J=8.0 Hz, 2H), 4.99 (s, 1H), 3.52-3.40 (m, 1H), 1.89 (dd, J=12.5, 5.0 Hz, 2H), 1.63 (dt, J=13.4, 4.1 Hz, 2H), 1.52 (dt, J=12.7, 4.1 Hz, 1H), 1.34-1.16 (m, 2H), 1.09 (tq, J=11.6, 7.7, 5.7 Hz, 3H). ¹³C NMR (101 MHz, CDCla) δ 153.7, 151.1, 129.1, 124.9, 121.6, 50.0, 33.1, 25.4, 24.7. FTIR (cm⁻¹) v: 3304.33, 2923.37, 2851.12, 1736.9, 1703.7, 1537.5, 1482.8, 1447.8, 1318.5, 1275.2, 1237.9, 1203.1, 1012.5, 962.7, 893.9, 775, 708.2, 686.4, 500.1, 443.

### Glipizide 1a

Glipizide 1a was afforded as a white solid, mp 208-209° C.; ¹H NMR (400 MHz, DMSO-d₆) δ 10.28 (s, 1H), 9.03 (d, J=1.5 Hz, 1H), 8.96 (t, J=6.1 Hz, 1H), 8.59 (s, 1H), 7.81 (d, J=8.0 Hz, 2H), 7.46 (d, J=8.0 Hz, 2H), 6.32 (d, J=7.8 Hz, 1H), 3.59 (q, J=6.9 Hz, 2H), 3.35-3.21 (m, 1H), 2.97 (t, J=7.2 Hz, 2H), 2.58 (s, 3H), 1.60 (ddt, J=25.3, 13.0, 3.8 Hz, 4H), 1.52-1.42 (m, 1H), 1.28-1.02 (m, 5H). ¹³C NMR (101 MHz, DMSO-d₆) δ 162.9, 156.8, 150.5, 144.9, 142.8, 142.3, 141.9, 138.2, 129.6, 127.7, 48.8, 35.5, 32.7, 25.4, 24.4, 21.7. FTIR (cm⁻¹) v: 3250.1, 1688.1, 1649.9, 1526.2, 1442.7, 1332.1, 1157.7, 1086.2, 1033, 902.8, 839.5, 686, 605.2, 576.7, 539.3, 443.7, 412.7.

### Glibenclamide 1b

Glipizide 1b was afforded as a white solid, mp 169-167° C.; ¹H NMR (400 MHz, DMSO-d₆) δ 10.30 (s, 1H), 8.26 (t, J=5.7 Hz, 1H), 7.85 (d, J=8.0 Hz, 2H), 7.66 (d, J=2.8 Hz, 1H), 7.49 (dd, J=8.5, 3.7 Hz, 3H), 7.15 (d, J=8.8 Hz, 1H), 6.34 (d, J=7.8 Hz, 1H), 3.80 (s, 3H), 3.56 (q, J=6.6 Hz, 2H), 3.35-3.22 (m, 1H), 2.94 (t, J=7.1 Hz, 2H), 1.69-1.52 (m, 4H), 1.52-1.43 (m, 1H), 1.29-1.09 (m, 4H), 1.07 (dd, J=11.2, 2.9 Hz, 1H). ¹³C NMR (101 MHz, DMSO-d₆) δ 163.6, 155.6, 150.6, 145.1, 138.3, 131.4, 129.5, 129.2, 127.2, 124.7, 124.9, 114.7, 56.6, 48.5, 35.1, 32.7, 25.4, 24.6. FTIR (cm⁻¹) v: 2929.9, 1713.2, 1614.9, 1518.7, 1340, 1276.4, 1243.6, 1156, 1093.1, 1011.2, 903.9, 818.9, 741.5, 684.6, 607.3, 572.8, 539.1, 439.4.

This above description of some of the illustrative embodiments of the invention is to indicate how the invention can be made and carried out. Those of ordinary skill in the art will know that various details may be modified thereby arriving at further embodiments, but that many of these embodiments will remain within the scope of the invention.

1. A flow synthesis process for producing a sulfonylurea compound of the Formula 1 or its pharmaceutically acceptable salts, 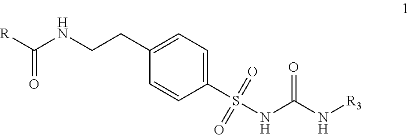
the process comprising the steps of:
a) preparing the amide of Formula 2 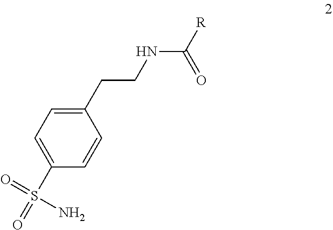
i) by activating a carboxylic acid of Formula 9 with a haloformate of Formula 8 in the presence of an organic base to produce an anhydride of Formula 5 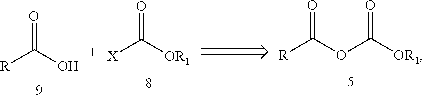
and
ii) reacting the anhydride of Formula 5 with the sulfonamide of Formula 4 to produce the amide of Formula 2, 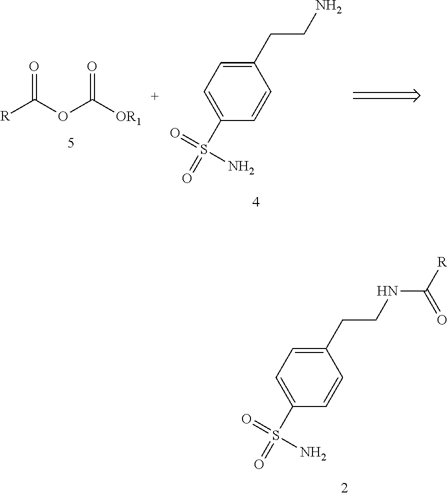
b) reacting the amide of Formula 2 with a carbamate of Formula 3 or isocyanate R₃—NCO, 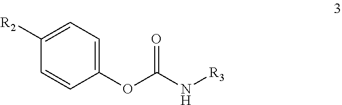
wherein the carbamate of Formula 3 is prepared through the reaction of R₃—NH₂ with the haloformate of Formula 7 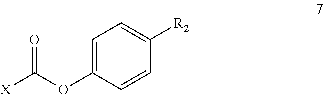
wherein:
R is selected from aryl or heteroaryl, which is unsubstituted or substituted with one or more occurrences of halogen, alkyl, and alkoxy;
R₁ is selected from phenyl, ethyl, or p-PhNO₂;
R₂ is selected from hydrogen and NO₂;
R₃ is a cyclohexyl; and
X is selected from Cl, Br, and I.

2. The process according to claim 1, wherein the process is a continuous multi-step process without the isolation of any intermediates.

3. The flow synthesis process according to claim 1, wherein R is selected from aryl or heteroaryl, which is substituted with one or more occurrences of halogen, alkyl, and alkoxy.

4. The flow synthesis process according to claim 3, wherein R is selected from aryl or heteroaryl, which is substituted with one or more occurrences of chloro, methyl, and methoxy.

5. The flow synthesis process according to claim 3, wherein R is selected from

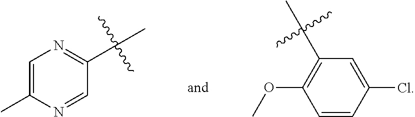

6. The flow synthesis process according to claim 1, wherein the organic base in step (a)(i) is selected from TBA, DBU, DIPEA, THA, TEA, and mixtures thereof.

7. The flow synthesis process according to claim 1, wherein the carboxylic acid of Formula 9 is dissolved in DMF.

8. The flow synthesis process according to claim 1, wherein the haloformate of Formula 8 is dissolved in acetonitrile.

9. The flow synthesis process according to claim 1, wherein the sulfonamide of Formula 4 is dissolved in DMF.

10. The flow synthesis process according to claim 1, wherein the reaction of the amide of Formula 2 with a carbamate of Formula 3 or isocyanate R₃—NCO in step (b) proceeds in the presence of an organic base selected from TBA, DBU, DIPEA, THA and TEA.

11. The flow synthesis process according to claim 10, wherein the organic base is DBU.

12. The flow synthesis process according to claim 10, wherein the amide of Formula 2 and the organic base is provided at a molar equivalent ratio of amide:base of about 1:1 to about 1:5.

13. The flow synthesis process according to claim 12, wherein the amide of Formula 2 and the organic base is provided at a molar equivalent ratio of amide:base of about 1:2 to about 1:3.

14. The flow synthesis process according to claim 1, wherein in the reaction of step (b) the amide of Formula 2 is dissolved in a solvent selected from DMF, acetonitrile, and mixtures thereof, and the carbamate of Formula 3 is dissolved in chloroform.

15. The flow synthesis process according to claim 1, wherein in the reaction of step (b) the amide of Formula 2 is provided at a concentration between about 0.01 M and about 0.5 M.

16. The flow synthesis process according to claim 1, wherein the reaction of step (a)(i) is thermally controlled to a temperature equal to about room temperature or less.

17. flow synthesis process according to claim 16, wherein the reaction of step (a)(i) is thermally controlled to a temperature of about room temperature.

18. The flow synthesis process according to claim 1, wherein the reaction of step (a)(ii) is thermally controlled to a temperature equal to about room temperature or less.

19. The flow synthesis process according to claim 18, wherein the reaction of step (a)(ii) is thermally controlled to a temperature of about room temperature.

20. The flow synthesis process according to claim 1, wherein the reaction of step (b) is thermally controlled to a temperature between about room temperature and about 150° C.

21. The flow synthesis process according to claim 20, wherein the reaction of step (b) is thermally controlled to a temperature of about 80° C.

22. The flow synthesis process according to claim 1, wherein the compound of the Formula 1 is selected from the compounds of Formula 1a or 1b and pharmaceutically acceptable salts thereof, 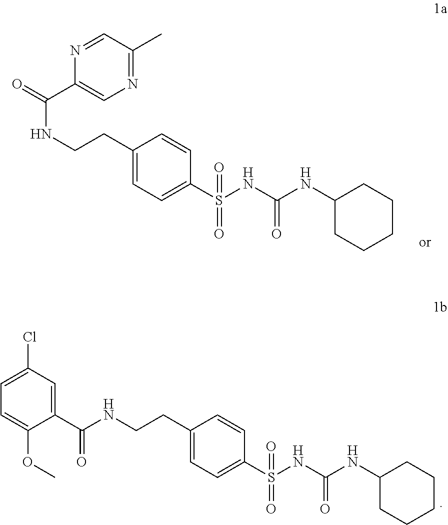 [SMILES: Cc1cnc(C(=O)NCCc2ccc(S(=O)(=O)NC(=O)NC3CCCCC3)cc2)cn1 ; COc1ccc(Cl)cc1C(=O)NCCc1ccc(S(=O)(=O)NC(=O)NC2CCCCC2)cc1]
